(19) United States
(12) Patent Application Publication
(10) Pub. No.: US 2007/0038472 A1
(43) Pub. Date: Feb. 15, 2007

(54) Systems and methods for managing clinical trials

(75) Inventors: Gerald E. Finken, Moorhead, MN (US); Kristina L. Schlecht, Portland, OR (US)

Correspondence Address: CROMPTON, SEAGER & TUFTE, LLC, 1221 NICOLLET AVENUE, SUITE 800, MINNEAPOLIS, MN, 55403-2420, US

(73) Assignee: Clinical Supplies Management, Inc.

(21) Appl. No.: 11/199,658

(22) Filed: Aug. 9, 2005

(51) Int. Cl. G06Q 10/00; A61B 5/00

(52) U.S. Cl. 705/2; 600/300

(57) ABSTRACT

Improved clinical trial methods including the use of subject monitors operating separately from standard clinical trial facilities and/or protocols. The subject monitors may independently or separately contact individual subjects to reinforce trial protocols, and observe compliance levels. Databases may be built and maintained for providing indications of the quality of a clinical trial. Clinical trial materials may be provided via the use of the independently operating subject monitors.

### FIELD

The present invention is generally related to the field of medicine. More particularly, the present invention is related to the field of clinical trial research.

### BACKGROUND

Medical innovations, from new drugs to new medical devices, are often researched and tested using a series of clinical trials. For example, there are typically four phases of clinical trials for a new drug. Phase I trials typically enroll a small sample of up to a few dozen study participants for testing of substance safety in humans. Phase I trials may have one or a small number of clinical investigation sites. Phase II trials typically enroll up to a few hundred study participants at several separate investigational sites, and include testing for safety, efficacy, and desirable dosing ranges. Phase III trials include relatively large scale testing on hundreds to thousands of participants at multiple sites to further identify and illustrate the efficacy of a desired dosing range, as well as continued verification of safety across a broader population. Phase IV of clinical testing is typically a post-approval trial performed during a conditional marketing period to prove continued efficacy and safety.

The FDA's March 2004 report, “Innovation, Stagnation, Challenge and Opportunity on the Critical Path to New Medical Products,” cites estimates that only about 8% of new medicinal compounds entering Phase I trials reach the market. As sample sizes increase from Phase I-Phase II and Phase II-Phase III, the cost of each successive trial increases. A product that successfully makes it to market may require upwards of five years and a billion dollars during clinical trials. Given such a low likelihood of success and the increasing costs of each trial phase, it is important that within clinical phases, problems and failures in testing/clinical processes be quickly identified.

### SUMMARY

The present invention, in an illustrative embodiment, provides methods of improving clinical trial efficacy. In an illustrative embodiment, subject monitors make contact with study subjects to query regarding their compliance and any concerns or difficulties. A record may then be kept of the number of interventions performed by the subject monitors to correct study subject behavior. In an illustrative example, the performance of individual sites and site monitors can be monitored, and the quality of the study can be assessed and trends can be established and monitored. A further embodiment includes a method where the subject monitor acts as an intermediary to arrange shipment of additional drug supplies to the subject. Another embodiment includes assessing study quality and providing the sponsor with an indication of how the study compares to other clinical studies.

### BRIEF DESCRIPTION OF THE DRAWINGS

FIG. 1 illustrates a configuration for a clinical trial;

FIG. 2 illustrates a modified clinical trial in accordance with an embodiment of the present invention;

FIG. 3 shows an illustrative method embodiment; and

FIG. 4 illustrates functional blocks of a database for another illustrative embodiment.

### DETAILED DESCRIPTION

The following detailed description should be read with reference to the drawings. The drawings depict illustrative embodiments and are not intended to limit the scope of the invention.

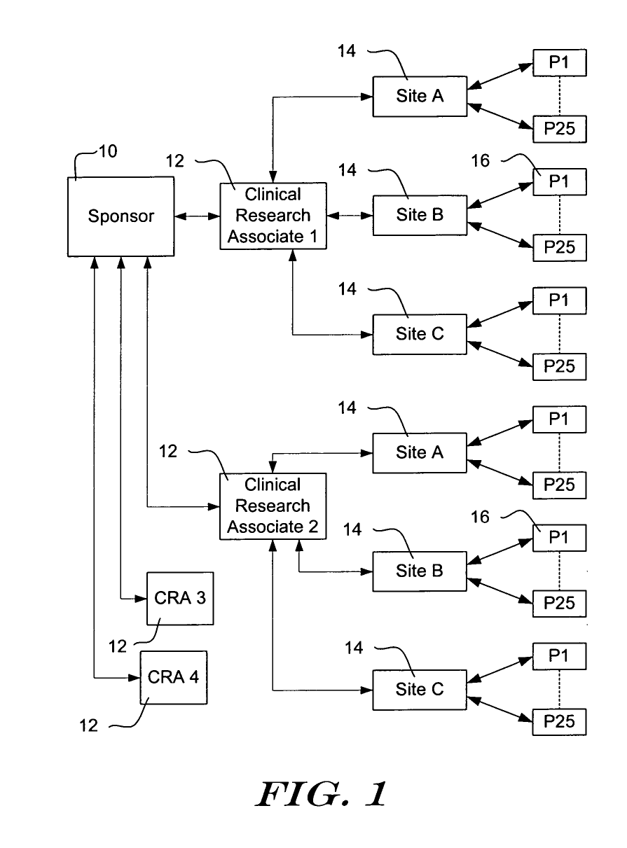

Historically, a clinical trial is sponsored by a sponsoring entity such as a private company, a medical or research institution, or a federal agency, or a by an entity established by a collaboration of such groups. An example configuration is shown in FIG. 1. The sponsoring entity 10 monitors the progress and data collection of the clinical trial by using one or more clinical research associates, or site monitors, 12 to oversee one or more investigational sites 14, with each site having a number of subjects 16. The sponsoring entity may include, coordinate, or involve such project management resources, vendors, labs, and contractors as are needed to provide services and products used in the study. The clinical research associates 12 monitor sites by making regularly scheduled visits and reviewing documentation.

Over time, the amount of information required to prove drug safety and efficacy in order to receive product approval has increased significantly. In response, some clinical research operations have been performed using electronic methods for gathering data and assuring subject compliance. Some examples of electronic methods used to address subject compliance include automated telephonic contacts and the use of electronic subject diaries. However, subject awareness of the accuracy and any changes in protocol instructions are not well tracked by these methods. Further, actual subject understanding of protocol instructions remains tied to individual clinical investigator and/or clinical research associates, whose time is divided not only among a plurality of locations and subjects, but whose time is often divided among multiple tasks and/or trials.

Various scenarios arise. For example, during early stage trials, subjects are likely to receive a greater level of one-on-one contact with clinical investigators, such that problems with instructions are less likely to interfere with trial results. However, this does not assure that the directions for use (DFU) given to subjects are themselves adequate. For another example, during later stage trials, administration difficulties can become systemic and difficult to root out, since those in charge of communicating with subjects to cause compliance are also the ones in charge of monitoring compliance. This is not to suggest that the clinical investigators manipulate compliance statistics, but more likely suggests that subjects are less likely to report non-compliance to those who selected them for a trial than to a third party.

In light of the above, the present invention is directed toward improving the process of performing clinical trials by taking certain tasks, which should each be performed consistently across a diverse subject population, out of the conventional system. In an illustrative embodiment, a clinical trial is directed by a sponsor using a number of clinical research associates each overseeing one or more clinical investigators, each clinical investigator having a number of study subjects. The clinical investigator may perform initial services including screening and selecting study subjects, providing initial instruction, and distributing an initial quantity of the study drug/dosage. Once the study is in place, the study investigator may be used to provide local services to the subject, for example, physically performing such tasks as taking blood samples, and/or performing physical examination. Additional monitoring, subject compliance assurance and interviewing, and/or drug distribution are then performed separately in an illustrative embodiment.

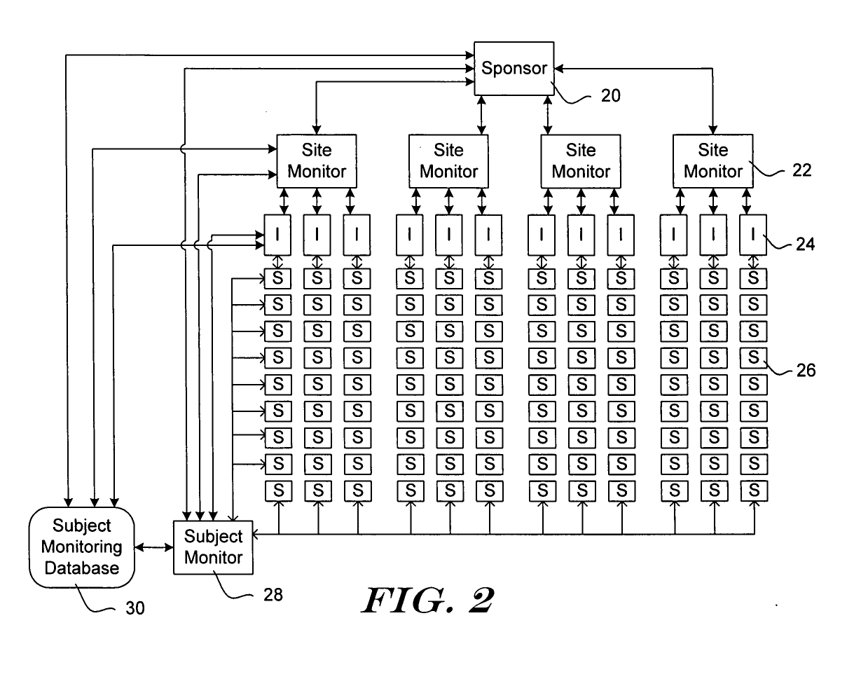

FIG. 2 illustrates a study performed in a manner consistent with an illustrative embodiment of the present invention. More particularly, a sponsor 20 works with site monitors 22 (sometimes using a clinical research associate but not necessarily), who in turn deal with individual clinical investigators 24 that are in charge of a number of subjects 26. However, in addition to, and separate from, the supervision and control of the site monitors 22 or individual investigators 24, is a subject monitor 28 who maintains a subject monitoring database 30.

There are multiple aims possible for the subject monitor 28. The following are illustrative examples, and a given subject monitor 28 in accordance with an embodiments of the present invention may or may not achieve each of the following aims. One aim is to provide individual contact and support to the subjects 26 of the clinical trial, providing an outlet for feedback and questions for the subjects 26, separate from the clinical investigators 24. Another aim is to gather the clinical data. Another aim is to provide feedback to the study sponsor 20 with respect to the performance of the site monitors 22 and investigators 24, the efficacy of automated telephonic contacts or electronic subject diaries, the adequacy of DFU, possible side effects or counter-indications, and/or ongoing subject compliance. In some embodiments, the data gathered by the subject monitor 28 takes the form of cognitive data related to the study subjects 26. The feedback from the subject monitor 28 may be provided in, or close to, real time, since the subject monitor 28 may have direct contact with all organizational layers of the clinical study. The subject monitor 28 may be, for example, one or a group of persons working to monitor the subjects 26. In some embodiments the subject monitor 28 includes one or more persons having a medical background including, for example, medical doctors, pharmacists, and/or nurses.

In some instances, the information gathered by the subject monitor 28 can supplement or contradict that gathered at sites by site monitors 22. This may be due to a lack of communication skills at the site, a “white-coat” syndrome, or simple luck (i.e. a study participant 24 may forget certain instructions or may figure out instructions after initial confusion). The actual cause does not change the simple fact that, by gathering added data in this separated manner, the trial can be analyzed for its effectiveness in properly delivering the tested substance to subjects and determining its safety, convenience, and efficacy. Study results gathered by the subject monitor may in fact contradict the results as indicated by others. If so, the gathered results may be compared to results gathered in more conventional manners to observe whether the study results are reliable.

By the use of subject monitors 28, the quality of the study itself, not just the compound being tested, can be analyzed. By having the subject monitor 28 in direct contact with the subjects, a variety of data can be gathered through interviews. One goal of such data is to provide compliance data with respect to the subjects and how well they use the clinical drugs. For example, the subjects may be interviewed to ask when they are taking the drug, how much they are taking, where it is stored, how much is available on-hand, how often the drug has been taken, etc. While subjects may themselves be hesitant to contact their physician or another practitioner involved in the study, when contacted directly it is more likely that those same subjects will voice problems, allowing the subject monitor to suggest or make physician contact. Another purpose of such data may be to ensure that all sites provide accurate and consistent training for subjects. For example, one site may provide DFU or interpret DFU in a way that is different than another site leading to inconsistent medication administration by subjects.

In some embodiments, the subject monitor can see what is going on across the clinical trial, rather than only within a single facility. This may allow additional insight into possible side effects, difficulty with automatic electronic contacts, or problems with DFU, for example.

In yet another embodiment, another role of the subject monitor is to help subjects understand their role in the clinical study. This may help the subjects understand the importance of complying and accurately reporting non-compliance. Further, an understanding of the subject's role in the clinical trial can improve their understanding of study procedures and informed consent.

In most clinical trials, the approach taken is to provide study results related to retained subjects. Some subjects will not be retained due to any number of reasons, including particularly non-compliance and inability to continue the study for health or other reasons. The use of study subject monitors to maintain statistics related to subject retention is another aspect of some embodiments.

In some embodiments, the subject monitor 28 may gather cognitive and/or subjective data with respect to the subjects 26. For example, the subject monitor 28 may ask open ended questions with respect to quality of life issues during the trial. The cognitive and/or subjective data may also be used to observe or detect trends in the data.

As shown in FIG. 2, the subject monitoring database 30 may be, in some embodiments, accessible to the sponsor 20 and/or one or more site monitors or clinical investigators 22. This access may be provided in any suitable form, including, for example, Internet Access, other electronic access, and/or printed reports. If desired, access may be limited to a read-only format. Access may also be limited to a “blind” format wherein only general information may be accessed, data related to individual subjects may be blanked out, or site monitor access may only relate to the wider study. The level of such access may vary depending on the study as well as dependent upon the needs or wants of the sponsor 20. The subject monitoring database 30 may also be accessible by, and may capture data from, the clinical investigators 24.

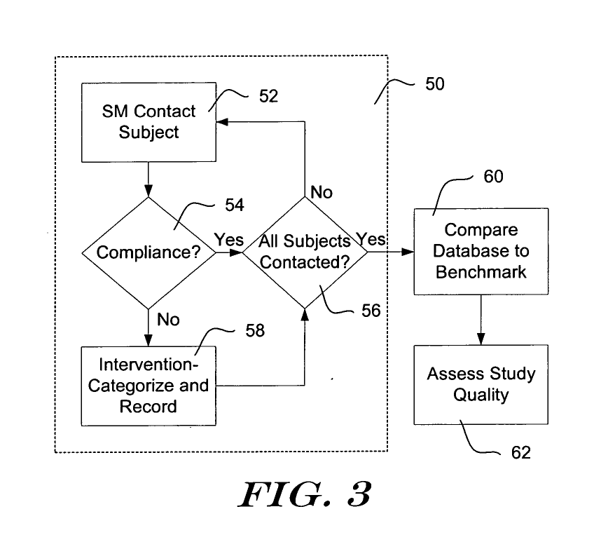

FIG. 3 shows in block form an illustrative embodiment. In this embodiment, the subject monitor is used to build a database related to clinical trials. The steps for building/populating the illustrative database are shown in block 50. The subject monitor contacts individual subjects, as shown at 52 and may ask a series of objective and/or subjective questions related to the subject's understanding of and compliance with study protocols, as shown at 54. If the subject is in compliance and understands the protocols adequately, the method continues by determining whether all subjects have been contacted, as shown at 56. If not, the method recycles by contacting the next subject. If the survey is complete at step 56, the method exits block 50.

If the subject is in some way non-compliant, the event is noted as an intervention, as shown at 58. Interventions may take a number of forms, and therefore may be categorized and recorded, as shown at 58. Some example interventions are occurrences wherein the subject has misunderstood the directions for taking a compound, the subject is out of medication and has failed to re-order, and/or the subject is suffering from some side effect which has gone unreported. When the intervention occurs, the subject may be instructed on proper directions for taking the compound, new medication may be ordered or sent, and/or the subject may be put in touch with a physician if necessary. The intervention may include directly notifying a physician if the subject is likely to be harmed. After the intervention, the method determines if the survey is complete, as shown at 56 and returns to step 52 if not completed, or exits block 50 if done.

Once out of block 50, the method continues by comparing the database to one or more benchmarks, as shown at 60, and assessing the study quality and/or trends. A variety of benchmarks may be used. For example, the number of interventions occurring during a study should drop off as the study continues forward. The number of interventions occurring in a given study of a given size (and perhaps within a given subject population) may be compared against one or more benchmarks including total interventions, persistence of interventions, or the like. Studies of different medications may be expected to show different results, compliance issues, or share other similarities. Within a study there may be a number of study sites; if one site illustrates a greater number of interventions than other sites, the site may be queried and results for that site may be questioned and/or discarded, or other analysis may occur.

During the course of the study, the subject monitors may be used to perform greater functions. For example, initial drug samples and directions may be given to study subjects by clinical investigators. Once the subject monitors make contact with the study subjects, however, further samples may be doled out by the subject monitors. The database may be used to generate a calendar of when study subjects should be ordering and/or receiving additional medication. The study monitor may also initiate the first drug shipment to the subject and bypass the clinical site all together. This is different than other database services that require the site to make contact and request medication shipments.

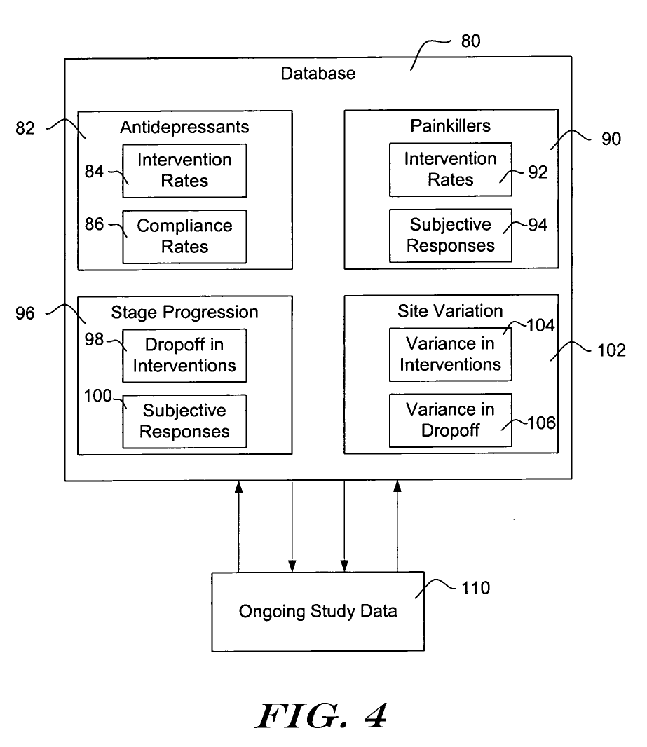

FIG. 4 illustrates functional aspects of a database that may be used and/or built in some illustrative embodiments. The database 80 is shown having category areas. For example, a category area for antidepressants 82 is shown as allowing access to data related to clinical studies involving antidepressant medications. Some data which may be accessed includes intervention rates 84 and compliance rates 86. This data 84, 86 may be gathered over a number of previous studies of antidepressant drugs that have been observed to provide benchmarks for expected intervention and compliance rates for other studies. A study falling outside a reasonable range for such rates, given the benchmarks from other studies, may be flagged as possibly indicating problems with DFU or other study parameters.

Another category area is for painkillers 90. In the category area for painkillers 90, data for intervention rates 92 and subjective responses 94 is shown. Subjective responses 94 may be indicative of certain cognitive data, for example, indicating how a drug is affecting subject moods. Again, data in these areas may be gathered from previous studies relating to painkillers.

Another category area is shown at stage progression 96, which may be indicative of trends or changes over time within a given study. For example, one data form available is data related to drop-offs in intervention 98. Another includes subjective responses 100 and how those responses change over time during a single study. The category area for stage progression 96 may include data gathered from across studies of different types of clinical treatment, for example, data from antidepressant and painkiller studies may be included.

FIG. 4 also shows a category area for site variation 102. This may include site-to-site variations including variance in interventions 104 and variance in drop-off 106 of interventions as time goes on. The category area for site variation 102 may provide data taken from a number of previous studies to indicate what is or is not “normal” for site-to-site variations within a given study. The various category areas and data types indicated in FIG. 4 are merely illustrative, and may be changed, expanded or substituted as desired.

In several illustrative examples, the database 80 is used for comparison to ongoing study data 110 being gathered during performance of another study.

For example, given a number of clinical studies already analyzed and placed in the database, a distribution of variances in interventions among a number of sites in a number of studies may be collected as data 104. This data may then be compared to an ongoing study to determine whether a problem with one (or more) of the study sites is indicated. For example, if a site falls more than two standard deviations away from the weighted average variance, when compared to the average for the ongoing study, it may be concluded that the site is performing poorly.

In another example, the rates for intervention for antidepressant studies may be gathered across a number of studies. If interventions in an ongoing study occur substantially more frequently than in similar studies performed with other antidepressant, the DFU may be revised and additional sites added to the study.

Yet another illustrative embodiment includes certifying a study. Suitable guidelines may be drawn in reliance upon the database. For example, within a given study of painkillers, intervention rates may be observed and used as ongoing study data 110. If the intervention rates remain below a selected level, when considered in comparison to the database data 92 for intervention rates in painkiller studies, the study itself may be certified as having used proper, well-understood directions. The selected level may be set in any suitable fashion and may even combine variables. For example, intervention rates overall may be required to remain below a set level (illustratively, less than 10% of all contacts required interventions) and affect less than a set number of subjects (less than 20% requiring more than one intervention during a study).

Interventions may be given weighted values. A point scale, for example, may assign a minor intervention (failing to take drug with food) a low point level (one point), a moderate intervention (taking drug in wrong dosage or at wrong time) a moderate point level (two points), and a severe intervention (failing to take the drug entirely) a high point level (three points). Cumulative points per subject, for example, could be another metric for measuring the quality of a study.

When one or more qualities of a study are measured, the results may be reported to a study sponsor, to sites and other actors within a study, and/or to a regulatory body interested in the study results. In some embodiments, guidelines may be set for what constitutes a “good” study, in terms, for example, of one or more of the above metrics. If a study exceeds the guidelines, the method may include indicating that the study exceeds expected or set study guidelines for one or more study quality metrics. The collected data may be added to the existing data set and further update the guidelines, if desired.

The specific areas of interest noted in FIG. 4 are provided for the purpose of illustration, and the present invention is not limited to these illustrative examples.

Those skilled in the art will recognize that the present invention may be manifested in a variety of forms other than the specific embodiments described and contemplated herein. Accordingly, departures in form and detail may be made without departing from the scope and spirit of the present invention as described in the appended claims.

What is claimed is:

1. A method of performing a clinical trial, the clinical trial directed by a sponsor utilizing site monitors to oversee one or more clinical investigators, each investigator having a number of study subjects, the investigator capturing study data from the subjects, the method comprising:
independently gathering data from study subjects separately from the investigators for review and input into a subject monitoring database; and
observing a quality of the study by analysis of the independently gathered data.

2. The method of claim 1 wherein the clinical trial includes the use of a patient-contact technology to gather standardized data from the patients, wherein the step of independently gathering data from study subjects is performed separately from the patient-contact technology.

3. The method of claim 1 further comprising reporting the study quality to the sponsor.

4. The method of claim 3 wherein the step of reporting the study quality includes identifying whether the independently gathered data agrees or disagrees with the previously captured study data.

5. The method of claim 3 wherein the step of reporting the study quality includes identifying whether the independently gathered data indicates whether the study exceeds a quality threshold.

6. The method of claim 1 further comprising independently distributing study drug to the study subjects independent of the investigator.

7. The method of claim 6 wherein an initial study setup is performed at each investigational site and initial drug supplies are provided to each subject at a corresponding investigational site, wherein subsequent samples are independently distributed.

8. The method of claim 7 wherein the subject monitoring database is used to monitor independent distribution of subsequent samples.

9. The method of claim 1 wherein the clinical trial is performed according to study guidelines developed with the approval of the sponsor, the method further comprising using independently gathered data recorded in the subject monitoring database to certify to the sponsor that the study has been carried out according to study guidelines.

10. The method of claim 1 wherein the step of gathering data from study subjects is completed as two way communication between the subject monitor and study subjects.

11. The method of claim 1 wherein the subject monitoring database further includes comparison data related to selected previous clinical trials, wherein the subject monitoring database is used to compare the clinical trial to the comparison data.

12. A method of performing a clinical trial, the clinical trial directed by a sponsor utilizing a network of clinical investigators, each investigator having a number of study subjects, the investigator distributing study drug to the study subjects and capturing study data from the study subjects, the method comprising: independently performing interviews with study subjects separate from the study sites; and independently delivering study drug to the study subjects separate from the study sites.

13. The method of claim 12 further comprising independently monitoring compliance and cognitive data separate from the study sites.

14. A method of performing a clinical trial, the method comprising: establishing a study subject network at a number of investigator sites using a first collection of personnel; performing study functions including study drug delivery and study result gathering using a subject monitoring group and sample delivery group for study subjects at a plurality of study sites, the subject monitoring group being separate from the first collection of personnel.

15. A method comprising:
contacting a number of subjects involved in a current clinical trial to determine whether individual subjects are experiencing the clinical trial in a correct manner and, if not, performing corrective action with respect to the individual subject and recording an intervention related to the individual subject; and
observing whether interventions occurring in the current clinical trial fall within an acceptable range by reference to a database containing information related to interventions in other clinical trials.

16. The method of claim 15 wherein the step of recording an intervention includes categorizing the intervention, wherein the step of observing whether interventions in the current clinical trial fall within an acceptable range includes comparing categorized interventions from other clinical trials to categorized interventions in the current clinical trial.

17. The method of claim 15 wherein the other clinical trials include a set of clinical trials related to testing of a substance having shared characteristics with a substance being tested in the current clinical trial.

18. The method of claim 15 wherein, within the clinical trial, a number of different sites are used, the individual subjects being spread across the number of different sites, wherein the step of recording the intervention includes noting which site the individual subject corresponds to.

19. The method of claim 18 further comprising evaluating the different sites by observing the number and types of interventions occurring in the current clinical trial for each of several different sites.
